(19) United States
(12) Patent Application Publication
(10) Pub. No.: US 2005/0164375 A1
(43) Pub. Date: Jul. 28, 2005

(54) Nucleic acid detection apparatus

(75) Inventor: Hisaaki Inoue, Himeji-shi (JP)

Correspondence Address: BRINKS HOFER GILSON & LIONE, P.O. BOX 10395, CHICAGO, IL, 60610, US

(73) Assignee: Sysmex Corporation

(21) Appl. No.: 11/040,981

(22) Filed: Jan. 21, 2005

(30) Foreign Application Priority Data
Jan. 23, 2004 (JP) 2004-16403

(51) Int. Cl. C12M 1/34

(52) U.S. Cl. 435/287.2; 435/288.7

(57) ABSTRACT

A nucleic acid detection apparatus is described that include: a nucleic acid detection apparatus for amplifying a nucleic acid in a reaction solution accommodated in a detection container and detecting the amplified target nucleic acid, the nucleic acid detection apparatus including a plurality of detectors having a light-emitting part for irradiating light on the detection container and a light-receiving part for receiving the light from the detection container, and a controller for adjusting the variance of photoreception signals of each light-receiving part to a uniform photoreception signal level for the plurality of detectors based on the variance of the detectors.

This application claims priority under 35 U.S.C. §119 to Japanese Patent Application No. 2004-016403 filed Jan. 23, 2004, the entire content of which is hereby incorporated by reference.

### FIELD OF THE INVENTION

The present invention relates to a nucleic acid detection apparatus for detecting nucleic acids.

### BACKGROUND

Nucleic acid detection apparatus detect nucleic acids by detecting a change in turbidity of a reaction solution in conjunction with a nucleic acid amplification reaction. For example, apparatuses of this type detect nucleic acid concentration by accommodating a reaction solution within a detection cell, heating the reaction solution to a predetermined temperature to amplify the nucleic acid within the reaction solution, and detecting the resultant turbidity of the reaction solution.

Detection of the turbidity of the reaction solution is accomplished, for example, by a photodetector provided with a light-emitting element and a light-receiving element. Specifically, light emitted from a light-emitting diode (hereinafter referred to as “LED”) functioning as a light-emitting element irradiates the reaction solution contained in the detection cell, and the light transmitted through the detection cell and the reaction solution is received by a light-receiving element. Then, the light received by the light-receiving element is detected and converted to an electrical signal by a photoelectric conversion means, and the turbidity of the reaction solution is detected in real time based on this electrical signal.

The above nucleic acid detection apparatus is provided with a plurality of photodetectors, and the photoreception sensitivity error must be adjusted among the photodetectors.

In such photodetectors, the LED light intensity requires temperature compensation since the light emission intensity of the LED changes due to the influence of temperature fluctuations in conjunction with the heating of the reaction solution. An example of art for temperature compensation of LED light intensity is disclosed in Japanese Laid-Open Patent Publication No. 2000-275281, which describes a photoelectric converter provided with a plurality of thermosensitive resistance elements having different thermoresistance coefficients for temperature compensation of LED light intensity, and temperature compensation circuit for selecting a suitable thermosensitive resistance element and switching the signal transmission path using a switch.

Since the photoreceptor element is also affected by temperature fluctuation in this photodetector, temperature compensation is also necessary for the photoreception signal output from the photoreceptor. An example of art for photoreceptor temperature compensation is disclosed in Japanese Laid-Open Patent Publication No. 08-122246, which describes a spectral analyzer for detecting measurement light diffracted into a spectrum by a spectrophotometric system at each wavelength by a photodiode array and measuring the intensity of the measurement light, wherein the spectral analyzer is provided with a temperature sensor for measuring temperature change of the photodiode array, and a correction means for correcting the measurement light intensity using signals from the temperature sensor. Prior art cited in this disclosure does not relate to nucleic acid detection apparatus.

In the nucleic acid detection apparatus described above, a plurality of photodetectors are provided so as to be capable of detecting the turbidity of a plurality of reaction solutions from perspectives such as work efficiency and the like. Such nucleic acid detection apparatuses are provided with an LED for each photodetector, and there is a difference in the light intensity produced due to manufacturing error during fabrication of the respective LEDs. Furthermore, the extent of the variance in the individual LEDs changes over time due to differences in frequency of use of the respective photodetectors, which results in differences in the amount of light emitted by the respective LEDs. In nucleic acid detection apparatus provided with a plurality of photodetectors, there is variance in the photoreception signals output from the photoreceptors based on the difference in the amount of light emitted by the LED of the individual photodetector. Moreover, in such nucleic acid detection apparatuses, a photoreceptor is provided for each photodetector, and differences arise in the light-receiving sensitivity of the photoreceptor due to manufacturing errors in the fabrication of each photoreceptor. Furthermore, the extent of the variance in the individual photoreceptors changes over time due to differences in frequency of use of the respective photodetectors, which results in differences in the light-receiving sensitivity of each photoreceptor. In nucleic acid detection apparatuses provided with a plurality of photodetectors, there is variance in the photoreception signals output from the photoreceptors based on the light-receiving sensitivity of the photoreceptor of the individual photodetector. These problems cannot be eliminated by applying the prior art to nucleic acid detection apparatus.

### SUMMARY

An object of the present invention is to provide a nucleic acid detection apparatus capable of high-precision nucleic acid detection when using any photodetector among a plurality of photodetectors.

A first aspect of the nucleic acid detection apparatus of the present invention is a nucleic acid detection apparatus for amplifying a target nucleic acid in a reaction solution accommodated in a detection container and detecting the amplified target nucleic acid, the nucleic acid detection apparatus including a plurality of detectors having a light-emitting part for irradiating light on the detection container and a light-receiving part for receiving the light from the detection container, and a controller for adjusting the variance of photoreception signals of each light-receiving part to a uniform photoreception signal level for the plurality of detectors based on the variance of the detectors.

A second aspect of the nucleic acid detection apparatus of the present invention is a nucleic acid detection apparatus for amplifying a target nucleic acid in a reaction solution accommodated in a detection container and detecting the amplified marker nucleic acid, the nucleic acid detection apparatus including a detector having a light-emitting part for irradiating light on the detection container and a light-receiving part for receiving the light from the detection container, a first controller for generating a correction value based on a reference photoreception signal output from the light-receiving part before amplification of the target nucleic acid begins, and a second controller for detecting a target nucleic acid based on the correction value and a photoreception signal output from the light-receiving part during amplification of the target nucleic acid.

A third aspect of the nucleic acid detection apparatus of the present invention is a nucleic acid detection apparatus for amplifying a target nucleic acid in a reaction solution accommodated in a detection container and detecting the amplified target nucleic acid, the nucleic acid detection apparatus including a plurality of detectors having a light-emitting part for irradiating light on the detection container and a light-receiving part for receiving the light from the detection container, a signal selector for inputting the photoreception signal from each light-receiving part, a controller detecting a target nucleic acid based on a photoreception signal selected by the signal selector during amplification of the target nucleic acid, and a memory for storing each correction value generated based on a second photoreception signal output from each light-receiving part by receiving the light from the reaction solution before amplification of the target nucleic acid, wherein the controller corrects the photoreception signal selected by the signal selector based on a correction value stored in memory, and detects the target nucleic acid based on the corrected photoreception signal.

### BRIEF DESCRIPTION OF THE DRAWINGS

FIG. 1A is a function block diagram schematically showing the structure of an embodiment of the nucleic acid detection apparatus;

FIG. 1B is a schematic perspective view showing the general structure of a nucleic acid detection apparatus;

FIG. 2 is a function block diagram schematically showing the structure of the control unit of the nucleic acid detection apparatus of FIG. 1A;

FIG. 3 is a function block diagram schematically showing the structure of the data logger of the control unit of FIG. 2;

FIG. 4 is a schematic perspective view showing the structure of the nucleic acid detection apparatus of FIG. 1B;

FIG. 5 is a schematic top plan view showing the structure of the nucleic acid detection apparatus of FIG. 1B;

FIG. 6 is a schematic cutaway view in perspective showing the structure of the output part of the nucleic acid detection apparatus of FIG. 1B;

FIG. 7 is a schematic view showing the layout of the detection part of FIG. 6;

FIG. 8 is a top plan view showing the layout of the detection part of FIG. 6;

FIG. 9 illustrates the change over time of the reaction solution turbidity detected by the detection part of the nucleic acid detection apparatus; and

FIG. 10 illustrates a calibration curve sowing the relationship between the amplification rise time and marker nucleic acid concentration using the nucleic acid detection apparatus.

### DETAILED DESCRIPTION OF THE DRAWINGS AND THE PRESENTLY PREFERRED EMBODIMENTS

An embodiment of the present invention is described hereinafter with reference to the drawings.

The nucleic acid detection apparatus of the present invention is described by way of example of a gene amplification detection device in the present embodiment. The gene amplification detection device is usable as an analyzer aiding the diagnosis of cancer metastasis in surgically excised tissue. In this case, genes (mRNA) of cancerous origin present in the excised tissue are amplified using the LAMP (loop-mediated isothermal amplification) method, and the cancer gene is detected by detecting the change in turbidity of the reaction solution generated in conjunction with the gene amplification. Details of the LAMP method are disclosed in U.S. Pat. No. 6,410,278, and are omitted from the following description of the embodiment.

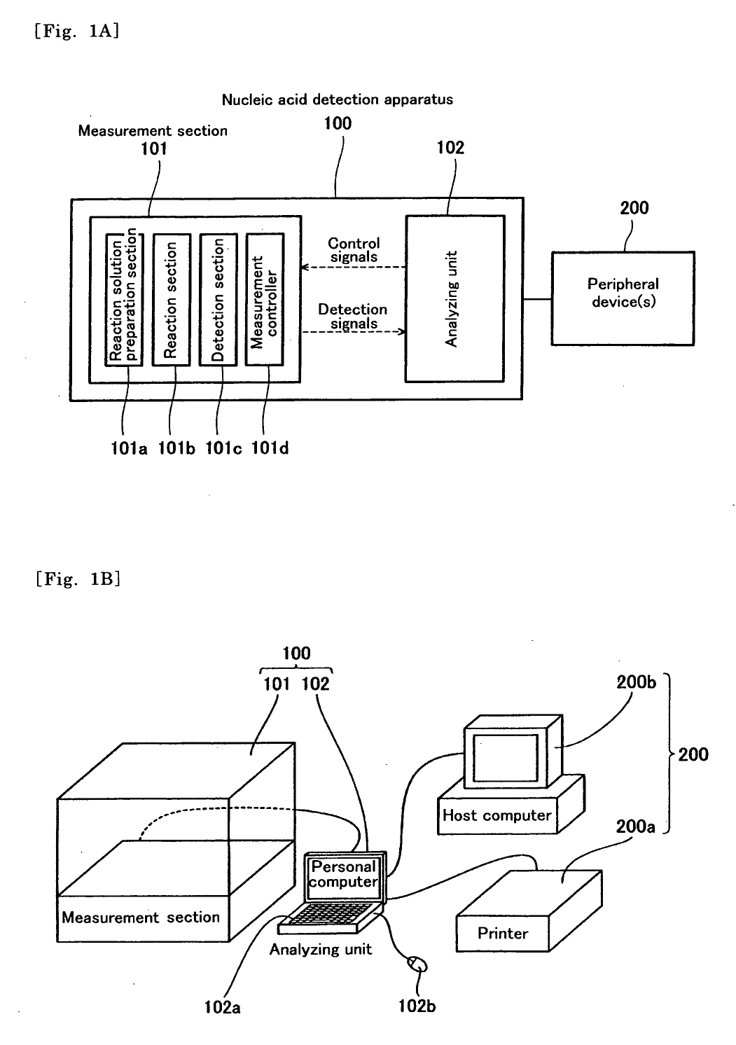

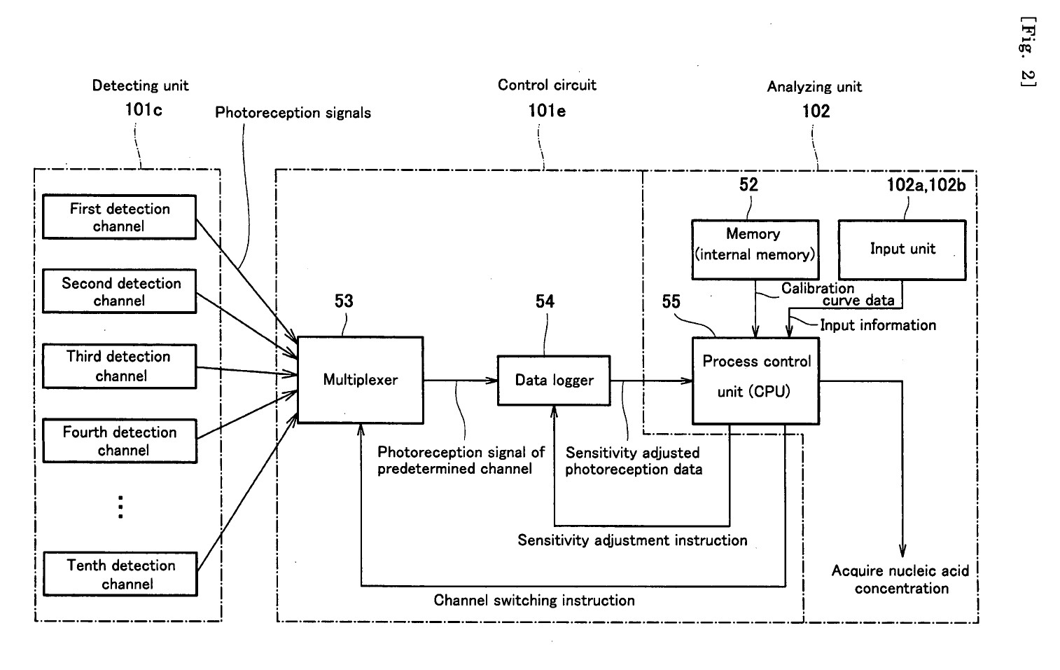

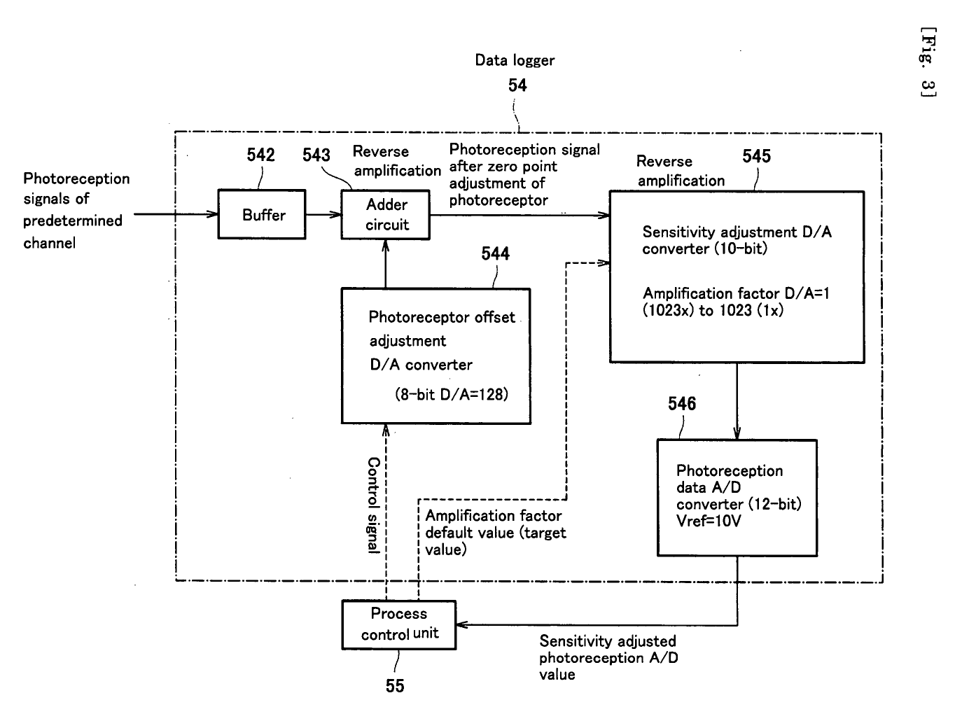

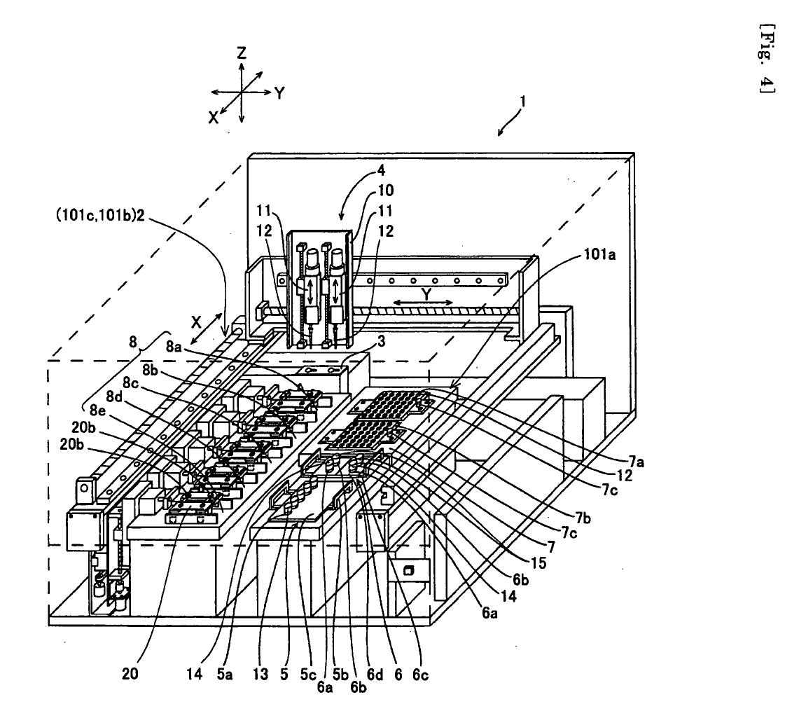

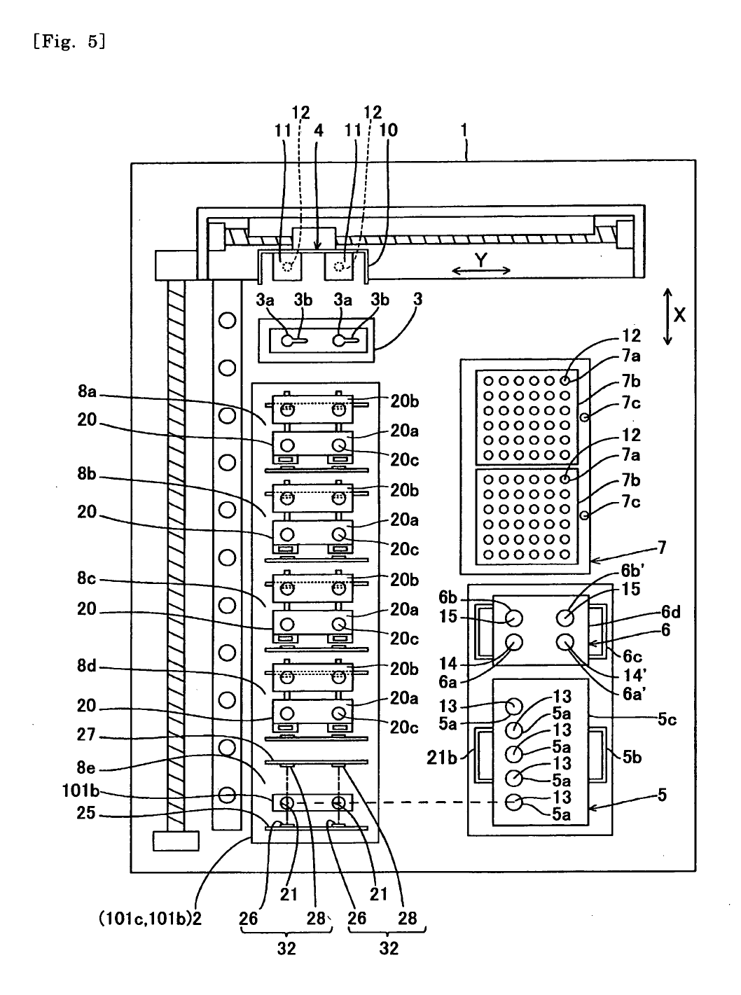

FIG. 1 is a schematic view showing the structure of an embodiment of v the nucleic acid detection apparatus of the present invention; FIG. 1(A) is a function block diagram schematically showing the general structure of the nucleic acid detection apparatus, and FIG. 1(B) is an example of the specific structure of the nucleic acid detection apparatus of FIG. 1(A). FIG. 2 is a function block diagram schematically showing the structure of the control unit of the nucleic acid detection apparatus of FIG. 1, and FIG. 3 is a function block diagram schematically showing the structure of the data logger of FIG. 2. Furthermore, FIG. 4 is a perspective view schematically showing the structure of the measuring unit of the nucleic acid detection apparatus of FIG. 1, and FIG. 5 is a top plan view of the measuring unit of FIG. 4.

As shown in FIG. 1(A), the nucleic acid detection apparatus 100 is provided with a measuring unit 101 and an analyzing unit 102. A suitable peripheral device 200 is provided for the nucleic acid detection apparatus 100. The measuring unit 101 includes a reaction solution preparation section 101a, reaction section 101b, detection section 101c, and measurement controller 101d. The analyzing unit 102 is provided with the structures shown in FIGS. 2 and 3.

As shown in FIG. 1(B), the measuring unit 101 housed within a case 1 (refer to FIG. 4) is connected through a communication line to the analyzing unit 102, which is a personal computer external to the case 1 and provided with an operation input unit (keyboard 102a and mouse 102b). A printer 200a and host computer 200b are connected to the analyzing unit 102 through a communication line as peripheral devices 200. The printer 200a prints various types of data, such as graphic data and text data and the like, output from the analyzing unit 102. The host computer 200b performs comprehensive management and processing of various types of data output from the analyzing unit 102.

As shown in FIGS. 4 and 5, the nucleic acid detection apparatus 100 has a structure which accommodates the measuring unit 101 within the case 1. The measuring unit 101 is provided with a reaction solution preparation section 101a, and a reaction detection unit 2 which includes a reaction section 101b and detection section 101c. The reaction solution preparation section 101a includes a dispensing mechanism 4, sample container holder unit 5, reagent container holder 6, chip holder 7, and chip disposal unit 3. The reaction detection unit 2 has a reaction detection block 8 including an array of detection cells accommodating reaction solution. The reaction detection block 8 includes five reaction detection blocks 8a, 8b, 8c, 8d, 8e arranged sequentially from the side near the chip disposal unit 3 along the X-axis direction indicated by an arrow in the drawing.

The dispensing mechanism 4 of the reaction solution preparation section 101a has an arm 10 constructed so as to be horizontally movable in the X-axis and Y-axis directions indicated by arrows in the drawing, and a pair of syringes 11 independently mounted on the arm 10 and constructed so as to be vertically movable along the Z-axis direction indicated by an arrow in the drawing. Pipette chips 12 are installed on the tips of the syringes 11.

Although omitted from the drawing, the two syringes 11 are respectively provided with a pump for suctioning and discharging reagent and sample solution, a motor as a drive source for the pump, liquid surface sensor, and pressure sensor. The functions of suctioning and discharging solution by the pump is accomplished by converting the rotational movement of the motor to a piston movement. The liquid surface sensor may be, for example, an electrostatic capacitance sensor that detects whether or not the tip of the pipette chip 12, which is formed of electrically conductive resin, makes contact with the liquid surface. The pressure sensor detects the pressure when the pump suctions and discharges the solution. Whether or not fluid suctioning and discharging is reliably accomplished is detected by the liquid surface sensor and the pressure sensor.

A removable sample container holder 5c provided with five sample container holes 5a and handle 5b is arranged in the sample container holder unit 5. The five sample container holes 5a provided in the sample container holder 5c are arranged in a row with predetermined spacing along the X-axis indicated by the arrow in the drawing. Sample containers 13 accommodating sample solution are placed in the five sample container holes 5a of the sample container holder 5c.

Nucleic acid detection in the nucleic acid detection apparatus 100 requires the creation of a calibration curve and detection of a negative control as previously mentioned. Therefore, a container accommodating a calibrator including a marker gene of a predetermined concentration as a standard for creating the calibration curve, and a container accommodating a negative control for confirming the apparatus and reagents are not contaminated are arranged in sample container holes 5a instead of containers accommodating sample solution.

A reagent container holder 6d provided with two primer reagent container holes 6a and 6a′, two enzyme reagent container holes 6b and 6b′, and a handle 6c is removably installed in a reagent container holder unit 6. The primer reagent container hole 6a and the primer reagent container hole 6a′ of the reagent container holder unit 6 are respectively arranged at a predetermined spacing along the Y-axis indicated by an arrow in the drawing. Furthermore, the enzyme reagent container hole 6b and enzyme reagent container hole 6b′ are respectively arranged at a predetermined spacing along the Y-axis direction indicated by an arrow in the drawing primer reagent containers 14 and 14′ respectively accommodating different types of primer reagents are respectively placed in the primer container holes 6a and 6a′, and enzyme reagent containers 15 and 15′ respectively accommodating different types of enzyme reagents are respectively placed in the primer container holes 6b and 6b′ of the reagent container holder unit 6.

In the present embodiment, a primer reagent container 14 accommodating cytokeratin (CK19) primer reagent is placed in the primer reagent container hole 6a, and an enzyme reagent container 15 accommodating CK19 enzyme reagent is placed in the enzyme reagent container hole 6b. Furthermore, a primer reagent container 14′ accommodating β-actin primer reagent is placed in the primer reagent container hole 6a′, and an enzyme reagent container 15′ accommodating β-actin enzyme reagent is placed in the enzyme reagent container hole 6b′.

Two racks 7b having holes 7a for accommodating 36 pipette chips 12 are removably arranged in the chip holder 7. Two release buttons 7c are provided on the chip holder 7. The rack 7b becomes detachable when the release button 7 is pressed. The pipette chip 12 is formed of an electrically conductive resin containing carbon, and a filter is installed within the pipette 12. This filter functions to prevent erroneous flow of solution to the syringe 11. The pipette chip 12 is subjected to electron beam irradiation when packed prior to shipment so as to avoid adverse effects on gene amplification caused by resolving enzymes such as human saliva and the like which might have adhered during the manufacturing process. Furthermore, the rack 7b housing the pipette chips 12 is stored with top and bottom covers installed before being placed in the chip holder 7.

The chip disposal unit 3 is provided with two chip disposal holes 3a for disposal of used pipette chips 12, and which are arranged with a predetermined spacing along the Y-axis direction indicated by an arrow in the drawing. A channel 3b having a width smaller than the chip disposal holes 3a is provided so as to connect the chip disposal holes 3a.

Blocks 8a through 8e of the five reaction detection blocks 8 of the reaction detection unit 2 include a reaction section 101b for performing an amplification reaction on the sample solution, and a detection section 101c for detecting the turbidity of the reaction solution. Details of the structure of one example of a reaction detection block 8 are described below.

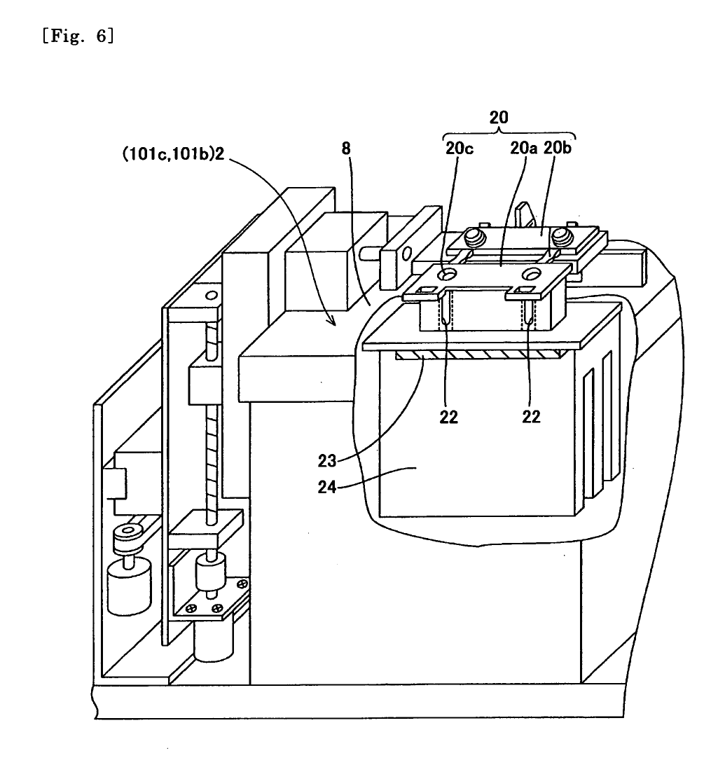

FIG. 6 is a cutaway perspective view schematically showing an enlargement of the structure of the reaction detection block 8. A detection cell hole 21 (refer to FIG. 5) for placement of the detection cell 20 containing a reaction solution is provided in the reaction section 101b of the reaction detection block 8. An irradiation channel 22 is provided within the detection cell 20 so as to allow light to enter the reaction solution and to allow light to be emitted from the solution. Furthermore, a Peltier module 23 and radiant heat sink 24 are provided within the detection cell 20 so as to heat and cool the reaction solution.

The detection cell 20 is formed by integratedly combining a cell member 10a formed of a heat-resistant transparent resin (for example, a crystalline olefin thermoplastic resin such as polymethylpentene (TPX) and the like) capable of transmitting light, and a cover member 20b formed of heat-resistant resin (for example, high density polyethylene). The cell member 20a forming the detection cell 20 is provided with two cells 20c, that is, spaces for accommodating reaction solution. The detection cell 20 is subjected to electron beam irradiation when packed prior to shipping so as to avoid adverse effects on gene amplification caused by resolving enzymes such as human saliva and the like which might have adhered during the manufacturing process.

The detection section 101c of the reaction detection block 8 is constructed to detect the turbidity of the reaction solution using light. Two LED light sources 26 are formed by surface-mounting blue LEDs having a wavelength of 465 nm corresponding to the two cells 20c on a substrate 25 arranged on one side surface of the detection cell 20 of the reaction section 101b. Two photodiode photoreceptors 28 are formed by mounting photodiodes corresponding to the LED photoemitter 26 on a substrate 27 arranged on the other side surface of the detection cell 20 so as to be opposite the substrate 25.

A detection unit 32 formed by one set of an LED photoemitter 26 and photodiode photoreceptor 28 is arranged in the detection section 101c of the reaction detection block 8 so as to correspond to each cell 20c. Accordingly, since the reaction detection unit 2 is provided with five reaction detection blocks 8a through 8e, a total of ten detection units 32 are provided. In this construction, ten detection channels are formed in the reaction detection unit 2 since one detection channel is formed by one detection unit 32.

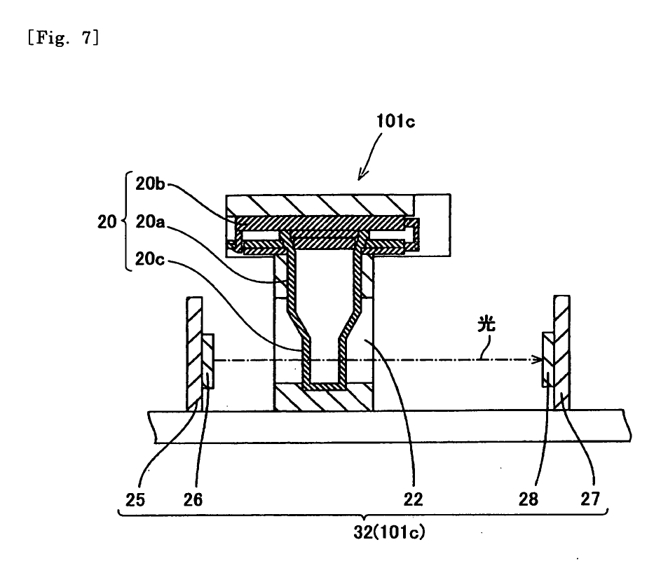

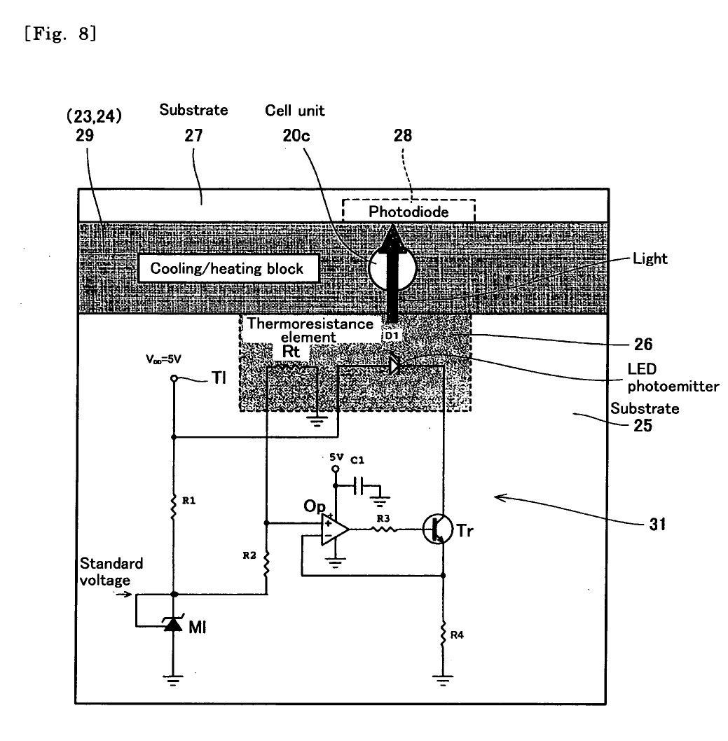

FIGS. 7 and 8 are schematic diagrams illustrating the structure of the individual detection units 32 (that is, individual detection channels) of the detection section 101c. FIG. 7 is a cross sectional view in the light advancement direction schematically showing the detection channel, and FIG. 8 is a top plan view schematically showing the structure of the detection channel.

As shown in FIGS. 7 and 8, in detection channel 32 which forms one detection channel, the substrate 25 provided with the LED photoemitter 26 and the substrate 27 provided with the photodiode photoreceptor 28 are arranged in opposition one to another and interposed between the detection cell 20 and a cooling/heating block 29 (refer to FIG. 8) which includes the Peltier module 23 and radiant heat sink 24, such that the light emitted from the LED photoemitter 26 toward the bottom of the cell 20c of the detection cell 20 is received by the photodiode photoreceptor 28. In the detection unit 32 of this construction, the presence or absence of the detection cell 20 can be detected, and the turbidity of the reaction solution contained in the cell 20c of the detection cell 20 can be detected in real time (monitoring).

In the nucleic acid detection apparatus 100 of the present embodiment which subjects a gene to an amplification reaction using the LAMP method, the LED photoemitter 26 heats and cools via the action of the cooling/heating block 29 so as to change the temperature during one reaction cycle from approximately 20° C. to approximately 65° C. within a predetermined time.

In each detection channel (each detection unit 32), the substrate 25 on the LED photoemitter side includes a temperature compensation circuit 31, which includes a drive source which functions as a source for supplying a drive current to the LED, for supplying a drive current from the drive source to the LED while adjusting the drive current supplied to the LED in accordance with the change in temperature of the LED photoemitter (refer to FIG. 8). The temperature compensation circuit 31 is provided with a thermosensitive resistor Rt which detects changes in temperature and linearly changes resistance value, that is, the resistor Rt functions as a temperature sensor.

During the gene amplification reaction time, the thermosensitive resistor Rt detects the change in temperature of the LED photoemitter 26, and the resistance value of the thermosensitive resistor Rt changes in accordance with the detected temperature change. Although the LED drive current output from the drive source is supplied to the LED through the temperature compensation circuit 31, the magnitude of the drive current supplied to the LED is regulated in accordance with the temperature since the resistance value of the thermosensitive resistor Rt in the temperature compensation circuit 31 changes in conjunction with the temperature as previously described. For example, when the temperature of the LED photoemitter 26 is high, the resistance value of the thermosensitive resistor Rt is regulated in accordance with this temperature and the drive power supplied to the LED is increased. Accordingly, since the drive power supplied from the drive source is increased, a decrease in the amount of light is suppressed and a constant amount of light is maintained even though the LED has a tendency to decrease the amount of emitted light at high temperatures.

In the temperature compensation circuit 31, an LED and npn transistor (hereinafter referred to simply as ‘transistor’) Tr are connected in series between a ground and a terminal T1 to which is applied a power source voltage VDD, such that a drive current corresponding to the collector current of the transistor Tr is supplied to the LED. Resistors R1 and R2 and the thermosensitive resistor Rt are connected in series, and together with a standard voltage setting element M1 are provided between a ground and the terminal T1. Since VDD changes due to the influence of load variation and noise and the like, a standard voltage is created by the operation of the standard voltage setting element M1, such as a Zener diode or the like, and the divided voltage of the thermosensitive resistor Rt and the resistor R2 corresponding to the standard voltage is input to the base of the transistor Tr through a differential amplifier (hereinafter referred to as ‘op-amp’) Op. The differential voltage of the divided voltage of the thermosensitive resistor Rt and the emitter voltage of the transistor Tr are input to the differential op-amp Op, and this differential voltage is amplified and input to the gate of the transistor Tr through a resistor R3. Accordingly, when the temperature of the LE rises, the resistance value of the thermosensitive resistor Rt increases, such that the divided voltage of the thermosensitive resistor Rt increases. Then, the voltage between the base and emitter of the transistor Tr increases, such that the collector current of the transistor Tr, that is, the drive current of the LED, increases. In this way the decrease in the amount of light emitted by the LED in conjunction with the rise in temperature is compensated. Therefore, in the photodiode photoreceptor 28, it is possible to suppress the generation of light reception errors caused by fluctuation in the amount of light emitted from the LED in conjunction with temperature change, and a constant amount of received light is maintained. A resistor R4 is an emitter resistor, which stabilizes the operation of the transistor Tr by negative feedback of the terminal voltage of this emitter resistor to the op-amp Op.

In the nucleic acid detection apparatus 100, since ten detection channels of each detection unit 32 respectively have the previously described LED temperature compensation circuit 31, the LED emission temperature compensation is performed in each detection channel.

The operation of the nucleic acid detection apparatus 100 is described below. The nucleic acid detection apparatus 100 performs a reaction solution preparation operation for preparing a reaction solution containing enzyme reagent and primer reagent and genes (mRNA) of cancerous origin present in surgically excised tissue, a gene amplification reaction operation for amplifying genes by the LAMP method, and a detection operation for detecting the nucleic acid concentration in the reaction solution by detecting the turbidity of the reaction solution resulting from the amplification of the gene. In the nucleic acid detection apparatus 100, when measurement is started, the gene amplification reaction and detection operations are performed.

First, a sample container 13 containing a soluble extract (hereinafter referred to as ‘sample solution’) prepared by preprocessing excised tissue by conventional methods, such as homogenization, filtration, and dilution and the like, is placed in the sample container hole 5a of the sample container holder 5c. A primer reagent container 14 containing CK19 primer reagent is placed in the primer reagent container hole 6a, and an enzyme reagent container 15 containing CK19 enzyme reagent is placed in the enzyme reagent container hole 6b. A primer reagent container 14′ containing β-actin primer reagent is placed in the primer reagent container hole 6a′, and an enzyme reagent container 15′ containing β-acting enzyme reagent is placed in the enzyme reagent container hole 6b′. Two racks 7b respectively accommodating 36 disposable pipette chips 12 are placed in the chip holder 7. At this time the position of the arm 10 of the dispensing mechanism 4 is a position a distance above the chip holder 7, specifically, above the chip disposal unit 3. In this way the two racks 7b may be easily placed in the chip holder 7. In the following description, the position above the chip disposal unit 3 is referred to as the ‘initial position’ of the arm 10. In each reaction detection block 8 of the reaction detection section 2, two cells 20c of the detection cell 20 are placed in the detection cell hole 21 of the reaction section 1I 1b.

Thereafter, necessary information such as measurement criteria and sample recording and light sensitivity level of the photodiode photoreceptor 28 (specifically, a target value described later) and the like are input by the operation input unit (specifically, the keyboard 102a and mouse 102b of FIG. 1(B)) of the analyzing unit 102. The input information is transmitted to the process control unit 55 (refer to FIG. 2). Then, a control signal for starting operation is output from the analyzing unit 102 to the measurement controller 101d of the measurement control section 101, and the operation of the nucleic acid detection apparatus 100 begins.

When the nucleic acid detection apparatus 100 starts operating, first, the arm 10 of the dispensing mechanism 4 is moved from the initial position above the chip disposal unit 3 to the chip holder 7. Then, at the chip holder 7, the two syringes 11 of the dispensing mechanism 4 are lowered in the Z-axis direction in the drawing. In this way the tips of the two syringes 11 are respectively press fitted into a top opening on the pipette chip 12 arranged in the chip holder 7, and the pipette chip 12 is automatically installed on the tip of each syringe 11.

The two syringes 11 with the installed pipettes 12 are raised in the Z-axis direction in the drawing, and the arm 10 of the dispensing mechanism 4 then moves in the X-axis direction in the drawing above the reagent container holder 6. Next, the two syringes 11 are lowered in the Z-axis direction in the drawing. In this way the tip of the pipette chip 12 installed on the tip of one syringe 11 is inserted to the liquid surface of the CK19 primer reagent in the primer reagent container 14, and the tip of the pipette chip 12 installed on the tip of the other syringe 11 is inserted to the liquid surface of the β-actin primer reagent in the primer reagent container 14′. Then, the CK19 primer reagent in the primer reagent container 14 is suctioned into one syringe 11, and the β-acting primer reagent in the primer reagent container 14′ is suctioned into the other syringe 11. When these primer reagents are suctioned, the contact of the tip of the pipette chip 12 with the liquid surface is detected by the liquid surface sensor (not shown in the drawing), and the pressure during suctioning by the pump (not shown in the drawing) is detected by the pressure sensor (not shown in the drawing. In this way suctioning can be verified.

After the respective primer reagents have been suctioned into the two syringes 11, the two syringes 11 are lifted in the Z-axis direction in the drawing. Then, the arm 10 of the dispensing mechanism 4 is moved above a predetermined reaction detection block 8 of the reaction detection unit 2. The arm 10 is first moved above the reaction detection block 8a nearest the chip disposal unit 3. In this movement, the arm 10 does not pass over the other reaction detection blocks 8b through 8e. Next, the two syringes 11 are lowered in the Z-axis direction in the drawing at the reaction detection block 8a at the moving end of the arm 10, and the pipette chips 12 installed on the tips of the two syringes 11 are inserted into the two cells 20c of the detection cell 20. Then, the CK19 primer reagent in one syringe 11 is discharged into one cell 20c, and the p-acting primer reagent in the other syringe 11 is discharged into the other cell 20c of the detection cell 20 using the pumps (not shown in the drawing) of the syringes 11. During the discharges, the contact of the tip of the pipette chip 12 with the liquid surface is detected by the liquid surface sensor (not shown in the drawing), and the pressure during suctioning by the pump (not shown in the drawing) is detected by the pressure sensor (not shown in the drawing in the same manner as during the suctioning operation. In this way discharge can be verified.

After the primer reagents have been discharged, the two syringes 11 are lifted in the Z-axis direction in the drawing. Thereafter, the arm 10 of the dispensing mechanism 4 moves along the X-axis direction in the drawing above the chip disposal unit 3. Then, at the chip disposal unit 3, the pipette chips 12 are discarded. Specifically, the pipette chips 12 are inserted into the two chip disposal holes 3a of the chip disposal unit 3 by lowering the two syringes 11 in the Z-axis direction in the drawing, and while in this state moving the arm 10 in the Y-axis direction in the drawing. In this way the pipette chip 12 is moved under the channel 3b. Then, a flange on the top surface (that is, the connector with the syringe 11) of the pipette chip 12 makes contact with the bottom surface of the bilateral sides of the channel 3b by raising the two syringes 11 in the Z-axis direction in the drawing, so as to receive a downward force from the top surface. In this way the pipette chips 12 are automatically detached from the tips of the two syringes 1 and discarded in the chip disposal unit 3.

After the primer reagents have been dispensed into the cells 20c of the detection cell 20 of the reaction detection block 8a as described above, enzyme reagents are dispensed in the cells 20c of the detection cell 20 of the reaction detection block 8a. Specifically, new pipette chips 12 are first installed on the tips of the two syringes 11 of the dispensing mechanism 4. Then, the arm 120 of the dispensing mechanism 4 is moved along the X-axis direction in the drawing above the reagent container holder 6. Then, the CK19 enzyme reagent in the enzyme reagent container 15 is suctioned into one syringe 11, and the β-acting enzyme reagent in the enzyme reagent container 15′ is suctioned into the other syringe 11. Thereafter, the two syringes 11 are moved above the reaction detection block 8a. Then, the CK19 enzyme reagent is discharged into the cell 20c containing the CK19 primer reagent of the detection cell 20 of the reaction detection block 8a, and the p-actin enzyme reagent is discharged into the other cell 20c containing the P-actin primer reagent. After the enzyme reagents have been discharged into the cells 20c, the pipette chips 12 are discarded in the chip disposal unit 3. The enzyme reagent dispensing operation and chip disposal operation are identical to the procedure for the primer reagents.

After the enzyme reagents have been dispensed into the cells 20c of the detection cell 20 of the reaction detection block 8a as described above, a sample solution is dispensed into the same cells 20c of the same reaction detection block 8a. Specifically, new pipette chips are first installed on the tips of the two syringes 11 in the same manner as when dispensing the primer reagents, and thereafter the arm 10 moves in the X-axis direction in the drawing toward the sample container holder 5. Then, the two syringes 11 are individually lowered in the Z-axis direction in the drawing and sample solution contained in one sample container 13 among the five sample containers present is suctioned into the two syringes 11.

In this procedure, one syringe 11 is moved to a position above one sample container 13 among the five sample containers 13 placed in the sample container holder 5. This syringe 11 is lowered and the sample solution in the sample container 13 is suctioned. Thereafter, this syringe 11 is raised, and the arm 10 of the dispensing mechanism 4 is moved along the X-axis direction in the drawing so as to position the other syringe 11 over the same sample container 13. This other syringe 11 positioned over the same sample container 13 is lowered and the sample solution is suctioned from the sample container 13, and subsequently the syringe 11 is raised. After the sample solution has been suctioned into each syringe 11 in this manner, the arm 10 of the dispensing mechanism 4 is moved above the reaction detection block 8a. Next, the two syringes 11 are lowered in the Z-axis direction in the drawing, and the sample solution is discharged into the two cells 20c of the detection cell 20 of the reaction detection block 8a. After the sample solutions have been discharged, the pipette chips 12 are discarded. The sample solution suctioning operation, arm 10 moving operation, and pipette chip 12 disposal operation are identical to the operations performed when dispensing the primer reagent and enzyme reagent.

When sample solution is discharged into the two cells 20c of the detection cell 20, the suctioning and discharge operations of the sample solution contained in the cells 20c are repeated a plurality of times using the pumps (not shown in the drawing) of the two syringes 11. In this way a reaction solution including the sample solution, enzyme reagent, and primer reagent contained in the two cells 20c are mixed.

A reaction solution including the primer reagent, enzyme reagent, and sample solution is prepared as described above. That is, a reaction solution including CK19 primer reagent, CK19 enzyme reagent, and sample solution is contained in one cell 20c of the detection cell 20 of the reaction detection block 8a, and a reaction solution including β-actin primer reagent, β-acting enzyme reagent, and sample solution is contained in the other cell 20c. When dispensing each of the prepared reaction solutions, the temperature of the liquids in the detection cell 20 are maintained at approximately 20° C. by regulating the temperature using the Peltier module.

After the reaction solutions are prepared as described above, the detection cell 20 is closed using the cover 20b. Thereafter, the reaction section 101b is heated using the Peltier module 23, such that the temperature of the reaction solutions in the detection cell 20 are raised from approximately 20° C. to approximately 65° C., and the gene amplification reaction via the LAMP method occurs for a predetermined time (18 min in the present example). The marker gene (mRNA) in the reaction solution is amplified by the reaction. The gene amplification reaction operation is performed in the nucleic acid detection apparatus 100 as described above.

In the gene amplification reaction described above, since magnesium pyrophosphate is generated as a byproduct of the amplification of the marker gene, the reaction solution becomes opaque as a by reaction byproduct such that the solution turbidity changes. In the detection section 101c of the nucleic acid detection apparatus 100 turbidity of the reaction solution in conjunction with the amplification of a marker gene is detected using the detection unit 32. That is, in the nucleic acid detection apparatus 100, a detection operation is performed in the detection section 101c at the same time the gene amplification reaction operation is performed in the reaction section 101b.

In the reaction solution turbidity detection performed in the reaction detection block 8a, a light beam (hereinafter referred to simply as ‘light’) having an approximate diameter of 1 mm is emitted from the LED photoemitter 26 of the detection unit 32, and this light irradiates the cell 20c of the detection cell 20 through the irradiation channel 22. Then, the light which is transmitted through the cell 20c and reaction solution contained therein (hereinafter referred to as ‘transmitted light’) is received by the photodiode photoreceptor 28 of the detection unit 32.

In the reaction detection block 8a, the detection cell 20 has two cells 20c, and reaction solution is accommodated in the cells 20c. Furthermore, a detection unit 32 is provided for each cell 20c. Therefore, two detection channels (in this example, referred to as a first channel and second channel) are formed in the reaction detection block 8a. In each detection channel transmitted light of the reaction solution contained in the cell 20c is detected in real time (monitoring) during the amplification reaction by the transmitted light being received by the photodiode photoreceptor 28. The light detected by the photodiode photoreceptor 28 is converted to an electrical signal and becomes a photoreception signal. The photoreception signal is sent to the measurement controller 110d whenever required.

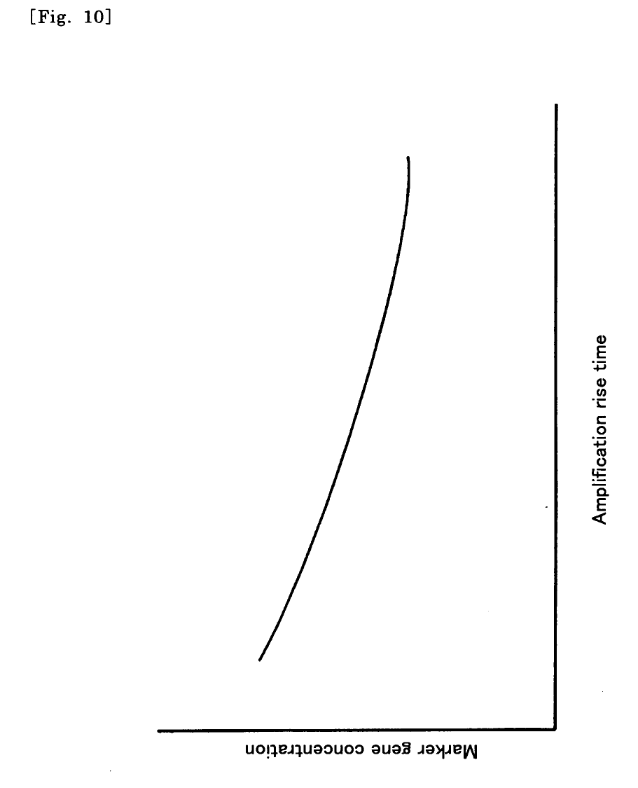

As shown in FIG. 2, the measurement controller 110d controls the reaction solution preparation section 101a, and detection section 101c, and the control circuit 101e of the measurement controller 101d includes a multiplexer 53 for normally receiving the photoreception signal of each channel received from the ten first through tenth detection channels and selecting the photoreception data of a desired detection channel and outputting the selected data to a data logger 54, and a data logger 54 for automatically generating time course photoreception data of the reaction solution of the selected detection channel using the photoreception signals of the desired detection channel. The analyzing unit 102 includes the operation input unit 102a and 102b, and memory 52, and process control unit 55. The analyzing unit 102 controls the multiplexer 53 and data logger 54 and obtains a turbidity by processing the photoreception data output from the data logger 54; and further includes the process control unit 55 which obtains the nucleic acid concentration in the reaction solution by comparing the obtained turbidity detection data with a calibration curve (specifically, the calibration curve shown in FIG. 10) stored in the memory 52.

In the control circuit 101e, the photoreception signals of the first and second channels output as described above are input to the multiplexer 53 as required for each channel. The multiplexer 53 functions as a switch for selecting a signal to send to the data logger 54 from among a plurality of input signals, and the data input path to the data logger 54 is switched by a control signal from the process control unit 55 such that only the photoreception signals of the required channel are input to the data logger 54.

The data logger 54 generates photoreception data for turbidity calculation from the photoreception signals of the desired detection channel (in this example, the first channel) input from the multiplexer 53.

Specifically, in the data logger 54, the photoreception signal from the first channel is input to a buffer 542, and input to a adder circuit 543, as shown in FIG. 3. In the buffer 542, the photoreception signal is amplified to prevent degrading, and in the adder circuit 543, the input photoreception signal is subjected to an adding process and reaction amplification process.

In the adder circuit 543, the photoreceptor signal of the first channel and an offset signal output from the photoreception offset adjustment D/A converter 544 are added, and the zero point adjustment of the first channel is accomplished by reaction amplification. The photoreceptor offset adjustment D/A converter 544 is controlled by control signals output from the process control unit 55.

The zero point adjustment of the first channel (offset adjustment) is a process for determining an offset value for setting the output signal of the photodiode photoreceptor 29 at [0] when the there is no light emission from the LED photoemitter 26, and adjusting the zero point based on the offset value. The process for determining the zero point adjusted D/A value (offset value) required for zero point adjustment is performed once when the power source is turned ON. The process control unit 55 calculates the zero point adjusted D/A value required for the photoreceptor offset adjustment D/A converter 544, and stores the value in the memory 52. Thereafter, the zero point adjustment is performed by the photoreceptor offset adjustment D/A converter 544 sensing the offset value to the adder circuit 543 based on the zero point adjustment D/A value stored in the memory 52.

A plurality of detection channels of the detection section 101c have their respective photodiode photoreceptors 28, and there is variation in the initial output values of the various photodiode element characteristics among the photodiode photoreceptors 28. As a result, the variation in the initial output values reduces the reliability and accuracy of the nucleic acid detection. In order to adjust the variation in the initial output signals among the various photodiode photoreceptors 28, the initial output signals of the photodiode photoreceptor 28 of each channel among the first through tenth detection channels are set uniformly at [0] regardless of the actual value. It is possible to adjust the variation in the element characteristics of the photodiodes among the detection channels by means of the zero point adjustment of the photodiode photoreceptors.

After this adjustment, the photoreception signal output from the adder circuit 543 is input to the sensitivity adjustment D/A converter 545. Then, the signal is reverse amplified at a predetermined amplification factor by the sensitivity adjustment D/A converter 545, and subjected to A/D conversion by the photoreception data A/D converter to create the photoreception data A/D value (transmitted light A/D). The predetermined amplification factor is an amplification factor at which a target photoreception sensitivity is realizable (that is, a target photoreception signal level for excellent detection, specifically, a target value described later), and is set beforehand for each detection channel by a sensitivity adjustment operation described later. The amplification factor is described in detail later.

Time-course change data of the reaction solution, that is, photoreception data A/D value (transmitted light A/D value) of the reaction solution, are created by processing the photoreception data in the data logger 54 as described above. The time-course change data are input to the process control unit 55. In the process control unit 55, the input transmitted light A/D value is subjected to processing to obtain turbidity (OD value) and create a turbidity detection value.

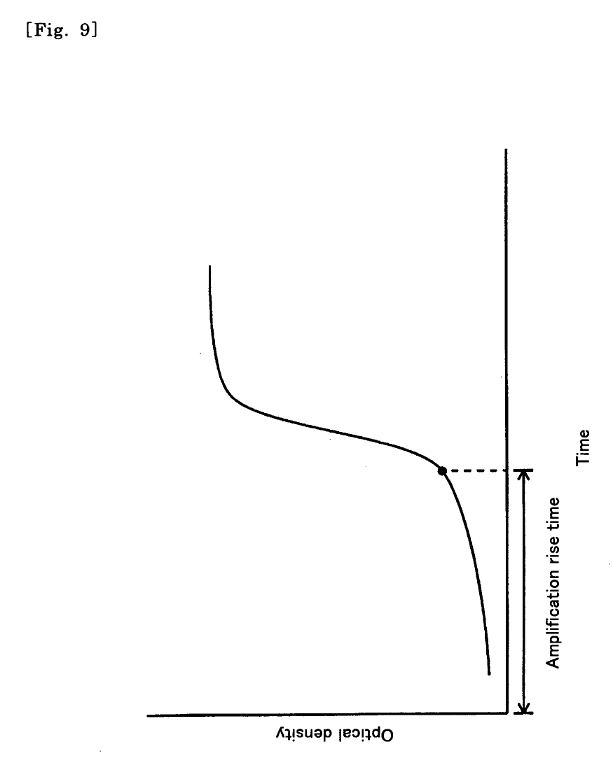

FIG. 9 shows the turbidity detection data of the reaction solution generated for the first detection channel, that is, the time-course change in the reaction solution turbidity in the first detection channel. In FIG. 9, turbidity (OD=optical density) is plotted on the vertical axis, and time is plotted on the horizontal axis. The turbidity detection data obtained by the process control unit 55 is subjected to the following processing in the process control unit 55.

In the process control unit 55, the amplification rise time is acquired which is the time until the target gene (mRNA) in the reaction solution attains a rapid replication number based on the change in the reaction solution turbidity, and target gene turbidity is acquired from the obtained amplification rise time and based on the calibration curve of FIG. 10 stored in the memory 52 of the analyzing unit 102 created from the measurement result of the calibration performed previously.

The calibration curve shown in FIG. 10 and stored in the memory 52 of the analyzing unit 102 has the amplification rise time plotted on the horizontal axis and the marker gene turbidity plotted on the vertical axis. It is clear from FIG. 10 that in general the shorter the rise time the higher is the turbidity of the marker gene. A container containing a calibrator including the marker gene of a predetermined concentration as a standard for generating a calibration curve, and a container containing a negative control for confirming that the apparatus and reagents are not contaminated are placed with predetermined frequency in the sample container holder 5, and the calibrator and negative control are subjected to the reaction solution preparation operation, amplification reaction operation, and detection operation identical to that of the reaction solution described above. The calibration curve shown in FIG. 10 can be created by the calibrator detection operation, and confirmation that the apparatus and reagents are not contaminated can be accomplished by the negative control detection operation.

After the nucleic acid concentration is acquired for the first detection channel of the reaction detection block 8a as described above, the process control unit 55 outputs a channel switching control signal to the multiplexer 53 so that the multiplexer 53 will output a photoreception signal of the second detection channel. In this way the same processes are executed for the second detection channel continuous to the first detection channel, and the nucleic concentration is obtained. In this way the nucleic acid concentration is acquired for the first and second detection channels of the reaction detection block 8a.

Next, in parallel with the detection operation performed in the reaction detection block 8a (that is, the first and second detection channels) in the nucleic acid detection apparatus 100, the reaction solution preparation operation, and subsequent gene amplification reaction operation and detection operation identical to those described above are performed in the reaction detection block 8b adjacent to the reaction adjacent block 8a. In this way nucleic acid concentration is obtained for the third and fourth detection channels in the same way as for the first and second detection channels. Then, in parallel with the detection operation in the reaction detection block 8b, a reaction solution preparation operation, and subsequent gene amplification reaction operation and detection operation identical to those described above are performed in the reaction detection block 8c adjacent to the reaction adjacent block 8b. In this way nucleic acid concentration is obtained for the fifth and sixth detection channels. The nucleic acid detection apparatus 100 sequentially performs the aforesaid series of operations in the five reaction detection blocks 8a through 8e, and obtains nucleic acid concentrations for the ten first through tenth detection channels of the reaction detection blocks 8a through 8e.

When the gene amplification reaction operation and detection operation are performed sequentially for each block from the reaction detection block 8a to the reaction detection block 8e, in one reaction detection block (for example, reaction detection block 8a), the gene amplification reaction operation and detection operation are performed simultaneously for two detection channels (in this case first and second detection channels). In the simultaneous detections in the first and second detection channels, the photoreception data (photoreception signals) detected in each of the first and second detection channels are input to the multiplexer 53 in parallel as required for each channel. Then, the photoreception data input path to the data logger 54 is switched at fixed intervals by the multiplexer 53 in accordance with control signals output from the process control unit 55. Furthermore, the amplification factor of the sensitivity adjustment A/D converter 545 is also switched in accordance with the detection channel simultaneous with the switching of the photoreception data. In this way the acquisition of nucleic acid concentration alternates between the first and second channels at fixed intervals. Since the switching of the detection channel and the processing of the photoreception data of the selected channel is performed instantly, photoreception data are apparently continuously obtained over time for one detection channel. Similarly, detection is performed simultaneously for two detection channels in the other reaction detection blocks 8b through 8e.

The processing in the previously described detection operation is performed instantaneously in the control circuit 101e of the measurement controller 101d in the analyzing unit 102. In this processing, channel switching from the first to the tenth detection channel occurs sequentially approximately every 100 microseconds, and the processing returns from the tenth detection channel to the first detection channel and is performed again.

The sensitivity adjustment operation at the measurement starting time is described by way of example below.

In the first and second detection channels of the reaction detection block 8a during the sensitivity adjustment operation, first, the sample solution is dispensed into the cell 20c of the detection cell 20 which contains primer reagent and enzyme reagent, and the a predetermined drive voltage is applied to the LED of the LED photoemitter 26, and light is emitted from the LED. Then, in the first and second detection channels, the light emitted from the LED irradiates the cell 20c, and light transmitted through the cell 20c is received by the photodiode photoreceptor 28. In this way the light received by the photodiode photoreceptor 28 of each detection channel is subjected to photoelectric conversion, and input to the multiplexer 53 of the analyzing unit 102 as photoreception signals for each channel.

Previously described processing for two channels is similarly performed for the third through tenth detection channels of the reaction detection blocks 8b through 8e.

After the photoreception signal of each channel has been input to the multiplexer 53, the photoreception signal of a predetermined channel (in this case the first channel) is output from the multiplexer 53 to the data logger 54 as previously described, and is subjected to adjustment relating to the zero point adjustment of the photodiode photoreceptor 28 in the adder circuit 543.

Thereafter, the photoreception signal is output from the adder circuit 543 to the sensitivity adjustment D/A converter 545. The photoreception signal target value for realizing excellent nucleic acid detection is set beforehand in the sensitivity adjustment D/A converter 545 through the process control unit 55. For example, when the control circuit 101e can only detect photoreception signal up to 10 V, then from the perspective of the upper limit of the detection range the target D/A value is set beforehand at 8 V. A target D/A value of 8V is equivalent to 3276 when converted to a digital value (that is, the target D/A value=3276).

The target D/A value is a common value of all detection channels.

In the sensitivity adjustment A/D converter 545, a predetermined default D/A value (hereinafter referred to as ‘sensitivity adjustment amplification factor D/A value’) is set beforehand through the process control unit 55 as the amplification factor D/A value for sensitivity adjustment.

The sensitivity adjustment amplification factor default D/A value is a value within the range of the amplification factor D/A values of the sensitivity adjustment D/A converter 545; the default D/A value is a value such that a photoreception signal amplified by the sensitivity adjustment amplification factor default D/A value is capable of appropriate sensitivity adjustment within the detectable range even when there is variation in the LED light emission amount; and the default D/A value may be optionally set insofar as the value satisfies this condition. The sensitivity adjustment amplification factor default D/A value need not be a value common to all detection channels, or may be a value common to all detection channels.

Since the amplification factor D/A value of the sensitivity adjustment D/A converter 545 is 1 (1023 times) to 1023 (I time) in the present embodiment, the sensitivity adjustment amplification factor default D/A value is set within this range. For example, when the sensitivity adjustment amplification factor default D/A value is [1] (1023 times), the amplified reception signal A/D value becomes 4095. Accordingly, in this case, an accurate photoreception A/D value is not obtained, and as a result suitable sensitivity adjustment is difficult. Therefore, in this case, the sensitivity adjustment amplification factor default D/A value is set at 1023 (1 time).

In the analyzing unit 102, when a photoreception signal is input from the adder circuit 543, the following sensitivity adjustment is performed using the sensitivity adjustment amplification factor default D/A value and the target A/D value. This sensitivity adjustment is described below using the first detection channel as an example.

First, in the sensitivity adjustment D/A converter 545, the photoreception signal (in this case, the photoreception signal of the first detection channel) input from the adder circuit 543 is reverse amplified by the sensitivity adjustment amplification factor default D/A value (=1023), and the amplified photoreception signal is subjected to A/D conversion in the A/D converter 546. The obtained photoreception signal A/D value (hereinafter referred to as ‘default photoreception signal A/D value’) is then output to the process control unit 55.

In the process control unit 55, the adjusted amplification factor D/A value is determined from the obtained default photoreception signal A/D value and the target A/D value (=3276).

The specific determination of the adjusted amplification factor D/A value is expressed in equation (1) below.
(adjusted amplification factor D/A value)=(sensitivity adjustment amplification factor default D/A value)×(default photoreception signal A/D value)÷(target A/D value) (1)

The sensitivity adjusted amplification factor D/A value obtained in the aforesaid sensitivity adjustment operation is extracted from the memory 52, and the photoreception signal is amplified in the sensitivity adjustment D/A converter 545 using this adjusted amplification factor D/A value. Accordingly, a photoreception signal is obtained which has a desired photoreception sensitivity at the target value.

As described above, the photoreception data (photoreception signals) of each detection channel output from the multiplexer 53 to the data logger 54 are switched at fixed times (for example, approximately every 100 microseconds). Therefore, after the photoreception data (photoreception signals) of the first detection channel are output from the multiplexer 53, the photoreception data (photoreception signals) of the second detection channel are output, and subsequently the photoreception data (photoreception signals) of the third through tenth detection channels are similarly and sequentially output. Thereafter, sensitivity adjustment is performed for each channel of the second through tenth detection channels in the same manner as for the first channel.

Then, an amplification factor D/A value, that is, and adjusted amplification factor A/D value, is obtained which conforms the photoreception signal A/D value output from the photoreception data A/D converter 546 to the target A/D value (=3276) for each channel by performing the sensitivity adjustment operation for the second through tenth detection channels. The adjusted amplification factor D/A value of each channel obtained in this way is stored in the memory 52 for each channel.

As described above, in the detection operation of the second through tenth detection channel, the obtained adjusted amplification factor D/A value is sent from the memory 52 to the sensitivity adjustment D/A converter 545 selected in accordance with the detection channel simultaneously with the channel switching by the multiplexer 53. Then, the photoreception signal is amplified in the sensitivity adjustment D/A converter 545 using the adjusted amplification factor D/A value. Accordingly, a photoreception signal at the desired sensitivity of the target value is obtained.

The sensitivity adjustment time among the detection channels gives rise to the relationship expressed in equation (2) by setting the adjusted amplification factor D/A value of each detection channel as described above. The reason equation (1) is obtainable is that the relationship in equation (2) is established immediately before and after sensitivity adjustment.

$$( amplification factor D / A value ) \times ( photoreception signal A / D value ) = ( a constant ) \qquad ( 2 )$$

From this it is possible to realize a uniform photoreception sensitivity at a target value among the detection channels even when there is variation in the photoreception signals among the detection channels.

As previously mentioned, although the amount of transmitted light (turbidity) of the reaction solution differs before amplification of the marker gene in each of the first through tenth detection channels, it is possible to normally maintain a uniform photoreception sensitivity for each detection channel even though the amount of transmitted light (turbidity) of the reaction solution differs before amplification of the marker gene by respectively determining the adjusted amplification factor D/A value conforming to a common target A/D value (=3276) by performing the sensitivity adjustment operation when measurement starts. Accordingly, it is possible to realize a uniform photoreception sensitivity at a target value without variations in photoreception signal levels even though the amount of transmitted light (turbidity) of the reaction solution differs before amplification of the marker gene in the detection channels.

It is also possible to monitor the condition of the LED photoemitter 26 using the sensitivity adjustment operation. For example, in the sensitivity adjustment operation performed during the rise time of the apparatus, the condition wherein a detection cell 20 is not placed in the reaction detection block 8 can be detected. In this case, it is possible to detect the generation of an error caused by a low amount of light emitted by the LED photoemitter 26 by setting the error range at an adjusted amplification factor D/A value set by the previously described sensitivity adjustment. In this case, the error may indicate a condition wherein it is difficult to perform suitable nucleic acid detection in the nucleic acid detection apparatus 100. Specifically, the adjusted amplification factor D/A value is temporarily stored in the memory 52 by the process control unit 55. The generation of an error caused by a low amount of emitted light from the LED photoemitter 26 can be detected by process control unit 55 retrieving and comparing the adjusted amplification factor D/A value stored in the memory 52 and a numeric value representing an error range.

The nucleic acid detection apparatus 10 of the present embodiment described above is capable of adjusting variance in photoreception signal levels caused by variation in the LED characteristics of the individual LED photoemitters 26 among a plurality of detection channels, variance in the photoreception signal levels caused by changes over time in the amount of light emitted by each LED, and variance in the photoreception signal levels caused on the light-receiving side using the control circuit 101e of the measurement controller 101d. Furthermore, variance in photoreception signal levels caused by variation in the photodiodes of the photodiode photoreceptors 28 in a plurality of detection channels can be adjusted by adjusting the photoreception data using the control circuit 101e of the measurement controller 110d. That is, variances caused on both the photoemitter side and the photoreceptor side can be adjusted by the control circuit 101e of the measurement controller 101d. Accordingly, detection accuracy and reliability are improved. Furthermore, sensitivity adjustment can be can be performed instantly in the control circuit 101e of the measurement controller 101d, such that sensitivity adjustment can be easily accomplished in real time during measurement. Moreover, the apparatus is realizable in compact form-factor and at low cost since sensitivity adjustment of a plurality of detection channels is accomplished by the control circuit 101e of the measurement controller 101d.

Detection accuracy and reliability are improved because variance in the amount of light emitted by the LEDs in accordance with the change in LED temperature of the LED photoemitters 26 is compensation by the temperature compensation circuit 31.

Since the amplification of a marker gene is accomplished using the LAMP method which performs direct amplification in a short time in the reaction section 101b, the marker gene can be effectively amplified and the time required for detection of the marker gene can be reduced. As a result, it is possible, for example, to perform the operations from sample placement to detection in approximately 30 min in the present embodiment. Since the temperature of the reaction section 101b changes from approximately 20° C. to approximately 65° C. within a predetermined time in the LAMP method, the temperature of the LED photoemitter 26 also changes. Accordingly, the effectiveness of the previously described temperature compensation circuit 31 is enhanced.

The present embodiment is one example of the present invention, however, the invention is not limited to this embodiment. For example, the number of detection channels of the reaction detection section is not limited to ten channels. Further, the arrangement and structure of the sample containers, reagent containers, and enzyme containers are not restricted. Although the reagent dispensation ad sample dispensation are both performed by a single dispensing mechanism in the present embodiment, a dispensing mechanism for dispensing reagent and a dispensing mechanism for dispensing sample may be provided separately.

The present embodiment has been described by way of example of amplification of a marker gene using the LAMP method, however, a marker gene may also be amplified by the polymerase chain reaction method (PCR method), and ligase chain reaction method (LCR method). The nucleic acid detection apparatus of the present invention is also applicable to detection of genes and mRNA other than cancerous origin.

The present embodiment has been described in terms of a detection section 101c, which detects reaction solution turbidity in a detection cell, including an LED photoemitter 26 and photodiode photoreceptor 28, however, a detection section including light-receiving units and light-emitting units provided with a light source means and a light-receiving means of alternative structure is also possible. For example, an alternative detection section may be provided with a light-emitting unit including an optical fiber connected to a lamp light source, and a light-receiving unit including a light sensor capable of receiving the light from the optical fiber of the light-emitting source. The present invention is even more effective when there is large variance in the characteristics of the individual light source means, and when there is large change over time in the amount of light emitted by the light source means.

The present embodiment has been described in terms of detecting a marker gene by providing a detection section for detecting the change in turbidity induced by amplification byproduct (specifically, magnesium pyrophosphate) within a detection cell 20, however, marker gene detection may be accomplished by methods other than turbidity detection, such as, for example, by detecting reagent bound to the marker gene using a predetermined detection device. In this case, examples of usable reagents include ethidium bromide, TaqMan probe and the like.

Although the LAMP method of detecting a marker gene is used in the above embodiment, the PCR method may also be used.

Although the measurement section and analyzing section are separate in the above embodiment, the measurement section and the analyzing section may be integrated.

In the nucleic acid detection apparatus 100 of the above embodiment, the reaction operation and detection operation are performed for each two channels in the reaction detection blocks 8a through 8e, however the reaction detection operation and detection operation may be performed simultaneously for all the reaction detection blocks 8a through 8e. In this case, the detection operation is performed simultaneously for ten detection channels, first through tenth channels, and the photoreception data (photoreception signals) of each channel are instantly input in parallel to the multiplexer 53 for each channel. Then, the multiplexer 53 switches the channels at fixed times, and the amplification factor of the sensitivity adjustment D/A value converter 545 is also switched for the switched channel as described previously.

In the nucleic acid detection apparatus 100 of the above embodiment, the sensitivity adjustment operation, that is, the process executed to conform the photoreception signal level to a target value, is performed when starting the gene amplification reaction operation and detection operation (that is, the start of measurement), and during the rise time of the nucleic acid detection apparatus 100 (specifically, when the power source is turned ON). However, as an alternative measurement starting time, sensitivity adjustment also may be performed when the sample solution is dispensed to the cell 20c which contains primer reagent and enzyme reagent during the reaction solution preparation process.

1. A nucleic acid detection apparatus for amplifying a target nucleic acid in a reaction solution accommodated in a detection container and detecting the amplified target nucleic acid comprising:
a plurality of detectors having a light-emitting part for irradiating light on the detection container, and a light-receiving part for receiving the light from the detection container; and
a controller for adjusting the variance of photoreception signals of each light-receiving part to a uniform photoreception signal level for the plurality of detectors based on the variance of the detectors.

2. The nucleic acid detection apparatus of claim 1, wherein the controller determines the amplification factor for adjusting the photoreception signal from each light-receiving part to a photoreception signal level, and determines the concentration of a target nucleic acid from photoreception data based on the amplification factor of the photoreception signal during nucleic acid concentration detection.

3. The nucleic acid detection apparatus of claim 2, wherein the product of the amplification factor and the photoreception data is constant.

4. The nucleic acid detection apparatus of claim 1, wherein the controller determines an offset value for adjusting the signal output from each light-receiving part to a predetermined standard value when a power source is turned ON and light is not emitted by any of the light-emitting parts.

5. The nucleic acid detection apparatus of claim 2 further comprising a memory for storing the amplification factor; and
wherein the controller stores the amplification factor determined when the power source is turned ON in the memory, and issues a warning when the amplification factor stored in memory is outside a predetermined range.

6. The nucleic acid detection apparatus of claim 1, wherein the controller performs adjustment before the start of target nucleic acid amplification.

7. The nucleic acid detection apparatus of claim 1 further comprising a temperature compensation circuit for correcting the change in the amount of emitted light from a light-emitting part in conjunction with the change in temperature of the detector.

8. The nucleic acid detection apparatus of claim 1, wherein the temperature compensation circuit comprises a thermoresistance element.

9. The nucleic acid detection apparatus of claim 1, wherein target nucleic acid amplification is accomplished by the LAMP method.

10. A nucleic acid detection apparatus for amplifying a target nucleic acid in a reaction solution accommodated in a detection container and detecting the amplified target nucleic acid comprising:
a detector having a light-emitting part for irradiating light on the detection container, and a light-receiving part for receiving the light from the detection container; and
a first controller for generating a correction value based on a reference photoreception signal output from the light-receiving part before amplification of the target nucleic acid begins; and
a second controller for detecting a target nucleic acid based on the correction value and a photoreception signal output from the light-receiving part during amplification of the target nucleic acid.

11. The nucleic acid detection apparatus of claim 10, wherein the reference photoreception signal is output from the light-receiving part by irradiating the detection container accommodating the reaction solution with light from the light-emitting part before starting amplification of the target nucleic acid.

12. The nucleic acid detection apparatus of claim 10, wherein an offset value is determined for adjusting the signal output from the light-receiving part to a predetermined standard value when the power source is turned ON and light is not emitted from the light-emitting part.

13. The nucleic acid detection apparatus of claim 12 further comprising a memory for storing the offset value; and wherein the control unit stores the offset value determined when the power source is turned ON in the memory, and generates the correction value based on the reference photoreception signal and the offset value stored in the memory.

14. The nucleic acid detection apparatus of claim 10, wherein the control unit determines a second correction value when the power source is turned ON, and issues a warning when the second correction value is outside a predetermined range.

15. The nucleic acid detection apparatus of claim 10 further comprising a temperature compensation circuit for correcting the change in the amount of emitted light from a light-emitting part in conjunction with the change in temperature of the detector.

16. The nucleic acid detection apparatus of claim 10, wherein the temperature compensation circuit comprises a thermoresistance element.

17. The nucleic acid detection apparatus of claim 10, wherein target nucleic acid amplification is accomplished by the LAMP method.

18. A nucleic acid detection apparatus for amplifying a target nucleic acid in a reaction solution accommodated in a detection container and detecting the amplified target nucleic acid comprising:
a plurality of detectors having a light-emitting part for irradiating light on the detection container, and a light-receiving part for receiving the light from the detection container;
a signal selector for inputting the photoreception signal from each light-receiving part;
a control unit detecting a target nucleic acid based on a photoreception signal selected by the signal selector during amplification of the target nucleic acid; and
a memory for storing each correction value generated based on a reference photoreception signal output from each light-receiving part by receiving the light from the reaction solution before amplification of the target nucleic acid;
wherein the controller corrects the photoreception signal selected by the signal selector based on a correction value stored in memory, and detects the target nucleic acid based on the corrected photoreception signal.

19. The nucleic acid detection apparatus of claim 18, wherein an offset value is determined for adjusting the signal output from the light-receiving part to a predetermined standard value when the power source is turned ON and light is not emitted from the light-emitting part.

20. The nucleic acid detection apparatus of claim 18, wherein the controller stores each offset value determined when the power source is turned ON in memory, and issues a warning when each offset value stored in memory is outside a predetermined range.
